(19) United States
(12) Patent Application Publication
(10) Pub. No.: US 2014/0154705 A1
(43) Pub. Date: Jun. 5, 2014

(54) ANTIBODIES TO OXIDIZED LDL

(71) Applicant: Victor Manneh, San Diego, CA (US)

(72) Inventor: Victor Manneh, San Diego, CA (US)

(21) Appl. No.: 13/587,914

(22) Filed: Aug. 16, 2012

(51) Int. Cl. G01N 33/92; C07K 16/18

(52) U.S. Cl. CPC G01N 33/92; C07K 16/18
USPC 435/7.92; 506/9; 530/388.25; 436/501; 422/69; 435/287.2

(57) ABSTRACT

A method is described for obtaining antibodies binding specifically to an epitope resulting from in vivo modification of a biomolecule or biomolecular complex, where the in vivo modification results from expression of a transgene in an animal. An example of such a biomolecule or biomolecular complex is LDL and an example of such a transgene is human myeloperoxidase. Resulting antibodies and methods of using the antibodies are also described.

### RELATED APPLICATIONS

NOT APPLICABLE.

### FIELD OF THE INVENTION

The present invention relates to methods of generating antibodies to in in vivo modified biomolecules.

### BACKGROUND OF THE INVENTION

The following discussion is provided solely to assist the understanding of the reader, and does not constitute an admission that any of the information discussed or references cited constitute prior art to the present invention.

As described in U.S. Pat. No. 7,575,873, atherosclerosis is a chronic inflammatory disease that results from hyperlipidemia and intereactions among a variety of environmental, metabolic, and genetic factors. Oxidation of low density lipoprotein (LDL) is important in the atherogenic process. Early research demonstrated that LDL acetylation greatly increased its uptake by macrophages. The increased uptake was found to be mediated by “scavenger receptors” distinct from the classical LDL receptor and which were not downregulated following uptake of oxidized LDL (oxLDL).

As a result of the very high macrophage uptake of oxLDL and associated lipid, the cells developed a characteristic foam-like appearance. The appearance of such foam cells is one of the early indicators of atherosclerotic disease. Foam cells accumulate within the intima (under the endothelial lining) of the vessel walls where they become unstable and form plaques, an indicator of more advanced atherosclerotic disease. Inflammatory conditions develop leading to the development of complicated lesions.

Substantial evidence has demonstrated the contributions of oxLDL to atherogenesis involving a number of mechanisms. The oxidation of polyunsaturated fatty acids in phospholipids of lipoproteins generates a range of breakdown products such as malondialdehyde (MDA), 4-hydroxynonenal (4-HNE), and other reactive moieties attached to oxidized phospholipids. Many of these intermediate products are highly reactive and can interact with lysine residues of associated proteins and phospholipids to generate various immunogenic adducts which occur in vivo.

In murine models of atherosclerosis, such as apo-E deficient mice (ApoE−/−) mice, atherosclerosis correlates with high titers of autoantibodies to oxidation specific epitopes of oxLDL.

It is generally accepted that the composition of atherosclerotic lesions, and especially the lipid, oxLDL, foam cell, and smooth muscle cell content of such lesions, determines their properties. Foam cells are often found in the sites of lesion that are susceptible to rupture. Activated macrophages recruited to clear the apoptotic and necrotic foam cells, as well as oxLDL, secrete factors that weaken the plaque. Human pathology studies have shown that atheromas containing a large necrotic core, thin fibrous cap and large numbers of macrophage/foam cells in the shoulder are more predisposed to plaque rupture and thrombosis. These lesions, which frequently appear as mild or moderate coronary stenoses in angiographic studies, are characterized pathologically as large atheroma with extensive lipid pools exceeding 40% of plaque areas. Angiography only provides a measure of arterial lumen, but fails to detect vessel wall pathology. Diagnostic methods that provide a measure of the overall extent of the atherosclerotic lesion, with an emphasis on oxLDL and lipid content, would therefore be desirable. Moreover, the lipid core of atheromas can be assumed to contain extensive oxidized lipids that accumulated within foam cells and were released when cells undergo necrosis and apoptosis.

Non-invasive detection of atherosclerotic lesions is currently not clinically feasible. The gold standard for diagnosing atherosclerosis is angiography which detects abnormal vessel lumen contours caused by encroaching atherosclerosis but does not directly identify abnormalities of the vessel wall. The widely recognized limitations of angiography include poor correlation with functional stenosis, interobserver and intraobserver variability, underestimation of the extent of disease because of diffusely atherosclerotic vessels, and arterial remodeling. B mode and ultravascular ultrasonography can detect intima/media thickening and calcification of vascular walls, but cannot clearly assess specific tissue characteristics. Electron beam computed tomography detects only calcium in vessel walls. Magnetic resonance imaging is still an investigational tool for the detection of plaque components.

Human studies have suggested that plaque rupture frequently occurs in nonangiographically significant lesions that contain abundant lipid-laden macrophages and large lipid pools within atheromas. Therefore imaging of atherosclerosis directed at lipid rich areas would be of value, not only in detecting the extent of lesion burden, but also in the detecting clinically silent but “active” lesions. Previous radioscintographic imaging agents have been limited by poor specificity, low in vivo uptake in atherosclerotic plaque, and slow elimination from the circulation, resulting in poor lesion/background ratios. Various imaging agents have been used including radiolabeled LDL, fragments of apolipoprotein B, autologous platelet and antiplatelet antibodies, non-specific antibodies and Fc fragments, hematoporphyrin derivatives, and anti-malonic acid monoclonal antibodies (Mabs).

OxLDL specific antibodies have been isolated from human and rabbit atherosclerotic lesions which contain tightly bound IgGs that recognize epitopes of oxLDL in vitro and stains atherosclerotic lesions in vitro. Mouse hybridoma cell lines have been generated for the production of Mabs against oxLDL and the antibodies were found to bind specifically to oxidized, rather than native phospholipids. However all of the antibodies previously described were monospecific, binding to only one form of oxLDL. The EO series of mouse Mabs described by Palinski et al. (1996), were able to bind either oxLDL or MDA-LDL, not both. Similarly, MDA2 and NA59, mouse Mabs described in other studies, bind MDA-LDL and HNE-LDL respectively. Most importantly, these mouse antibodies are limited in their usefulness for human applications in vivo as they elicit an immune response that prohibits their repeated administration.

Hybridoma technology, which is widely used in generating murine Mabs, is less successful in producing human hybridomas. Epstein Barr Virus (EBV) may be used to immortalize human lymphocytes, however due to the wide variety of neoepitopes in oxLDL, acquisition of human Mabs to many different epitopes would be arduous. Furthermore, clones derived by this technique are frequently unstable and low secretors. Additionally, the EBV-transformants produce IgM antibodies, while anti-oxLDL antibodies can be both IgG and IgM isotypes.

Phage display combinatorial library technology provides a useful method to generate human Mabs (Barbas and Lerner, 1991; Huse, et al., 1989). The libraries made from lymphocyte mRNA may consist of up to 10⁸ recombinants of monoclonal Fab repertoires. By displaying the library on a filamentous phage surface and panning against a model epitope, monoclonal antibodies (e.g., Fab or scFV) can be selected and analyzed for their immunological properties and biological activities. Fabs and scFv are advantageous for use in both therapeutic and diagnostic methods as they can be produced in large quantities inexpensively and they are innately non-immunogenic. Additionally, they are not whole antibody molecules which can initiate a cascade of immune responses upon binding to their antigen.

### SUMMARY OF THE INVENTION

The present invention concerns a method for obtaining antibodies which specifically bind to in vivo modifications of biomolecules. The method involves using a transgenic animal in which the transgene encodes a product which results in the in vivo modification of the biomolecule creating an immunogenic epitope. This approach is especially advantageous for obtaining useful antibodies binding to modified biomolecules which undergo varied modification in vivo.

Thus, a first aspect of the invention concerns a method for producing antibodies specific for in vivo-modified biomolecules. The method involves screening monoclonal antibody clones for specific binding activity to an in vivo modification epitope on a biomolecule or biomolecular complex, where the antibody clones are derived from a transgenic animal expressing a transgene. Expression of the transgene results in the in vivo modification or an increase in the level of the in vivo modification. Antibody clones are identified from the screening which have the specified specific binding activity.

In particular embodiments, the biomolecule or biomolecular complex is low density lipoprotein (LDL); the transgene is human myeloperoxidase and the modified biomolecule is a component of oxidized low density lipoprotein (oxLDL); the transgenic animal is a transgenic mouse, e.g., expressing human myeloperoxidase; the transgene is a human gene, e.g., in a mouse, rat, rabbit.

In certain embodiments, the antibodies binding specifically to the in vivo modification are selected from a plurality of hybridoma cell lines derived from the transgenic animal; the antibodies binding specifically to the in vivo modification are selected by panning a plurality of clones in a phage display library or yeast display library; the selected antibody is an intact antibody; the selected antibody is an antibody fragment, e.g., an Fab fragment, an Fab′ fragment, a scFv fragment, or a sdFv fragment.

In particular embodiments, the antibody preferentially binds to a plurality of different forms of oxidized LDL, including at least one of mpo-LDL, MDA-LDL, and cu-LDL as compared to binding to unmodified LDL; the antibody preferentially binds to mpo-LDL and MDA-LDL; the antibody preferentially binds to mpo-LDL and cu-LDL as compared to binding to unmodified LDL; the selected antibody has at least 1.5, 1.7, 2.0, 2.2, 2.5, 3.0, 4.0, 5.0, 7.0, or 10.0 times higher affinity for a form of oxidized LDL than for unmodified LDL.

In a related aspect, the invention concerns an isolated monoclonal antibody which is an intact monoclonal antibody or an antibody fragment derived from a transgenic animal expressing a transgene, where expression of the transgene results in in vivo modification of a biomolecule or biomolecular complex, and the antibody binds specifically to an epitope containing at least a part of the in vivo modification.

In particular embodiments, the monoclonal antibody includes a variable sequence preferentially binding to a modification epitope resulting from expression of human myeloperoxidase in a non-human animal, e.g., a mouse.

In particular embodiments, the monoclonal antibody is as described for the preceding aspect of an embodiment thereof or otherwise described herein for the present invention.

Another related aspect of the invention concerns a kit for determining the presence or amount of oxidized LDL (oxLDL) in a sample. The kit includes at least one antibody that specifically binds to an oxidized epitope in oxLDL, where the antibody is derived from a transgenic animal expressing human myeloperoxidase and preferentially binds to mpo-LDL rather than un-modified LDL.

In certain embodiments, the kit includes a plurality of single-use aliquots of the antibody, e.g., single-use aliquots in amounts suitable for use in separate runs of an assay for determining the presence or amount of oxLDL in a sample; the kit includes a plurality of different antibodies as specified for this aspect; the kit includes a plurality of lateral flow assay devices, wherein a single use aliquot of the antibody is present in each of the assay devices.

In embodiments in which phage display of antibodies is used, the method can also include isolating nucleic acids encoding antibodies from the transgenic animal, cloning nucleic acid (usually DNA) encoding antibodies (usually antibody fragments) into an appropriate phagemid, growing the phagemids in a corresponding host bacterial strain, purifying phagemids from the bacterial culture, panning pools of phagemids for specific binding activity to oxLDL (usually with binding to normal LDL as a control), selecting phagemid pools which show a desired binding activity (e.g., greater binding to a particular form(s) of oxLDL than to normal LDL or binding to normal LDL essentially the same as to oxLDL (or preferentially binding to normal LDL over oxLDL), isolating phagemids encoding antibodies having desired binding activities from phagemid pools which showed the desired activity during the panning, cloning nucleic acid from phagemids encoding antibodies having desired activity into a suitable expression vector, expressing the antibodies using the expression vector, and/or isolating, purifying, or enriching the antibodies expressed using the expression vector. In many cases, the expression vector will be an expression plasmid phagemid, or Yeast Artificial Chromosome (YAC) and the antibody will be expressed in a bacterial or yeast cell, but in some cases DNA encoding the antibody will be inserted chromosomally into a selected expression cell and expressed from that chromosomal insert.

Similarly when a yeast display system is used, the method can include common steps involved in use of such systems, e.g., expressing intact antibodies or antibody fragments in a yeast display strain, screening the yeast display strains identifying strains expressing antibodies exhibiting desired binding activity (e.g., greater binding to a particular form(s) of oxLDL than to normal LDL or binding to normal LDL essentially the same as to oxLDL (or preferentially binding to LDL over oxLDL), cloning nucleic acid encoding antibodies having desired binding activity into a suitable expression vector, expressing the selected antibodies having desired binding activity using the expression vector, and/or isolating, purifying, or enriching the antibodies expressed using the expression vector. Similar to use of phage display, in many cases, the expression vector will be an expression plasmid, phagemid, or Yeast Artificial Chromosome (YAC) and the antibody will be expressed in a bacterial or yeast cell, but in some cases DNA encoding the antibody will be inserted chromosomally into a selected expression cell and expressed from that chromosomal insert.

In particular embodiments, the antibody is as specified for the first aspect above or an embodiment thereof.

A method for determining the presence or amount of oxidized LDL (oxLDL) in a sample, where the method involves contacting the sample with at least one antibody which specifically binds to oxLDL. The at least one antibody is/are derived from a transgenic animal(s) expressing a transgene which results in in vivo oxidation of LDL. Binding of at least one of the antibodies to LDL is detected as an indication of the presence or amount of oxLDL in the sample.

In particular embodiments, the transgene is human myeloperoxidase.

In certain embodiments, the antibody is as specified for the first aspect above or an embodiment thereof.

Additional embodiments will be apparent from the Detailed Description and from the claims.

### BRIEF DESCRIPTION OF THE DRAWINGS

FIG. 1 shows reactions involving myeloperoxidase (mpo).

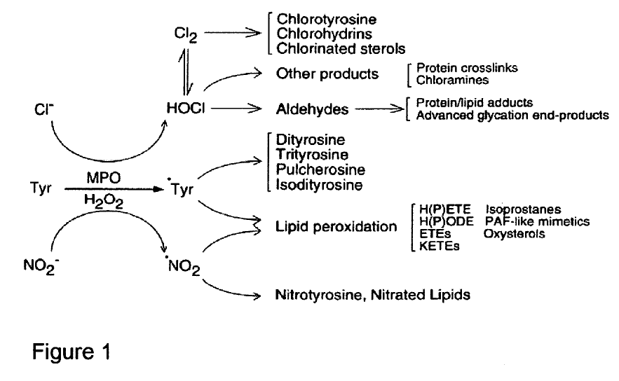

### DETAILED DESCRIPTION OF THE PREFERRED EMBODIMENTS

### Introduction

The present invention is directed to the identification and use of antibodies that recognize modification epitopes on a biomolecule or biomolecular complex resulting from in vivo modification in a transgenic animal expressing a transgene. The expression of the transgene results in the in vivo modification or an increase in the level of the in vivo modification. A particularly advantageous example is the identification of antibodies recognizing naturally occurring oxidized low density lipoproteins (LDL) and/or high density lipoproteins (HDL). Thus, the following discussion will emphasize and be illustrated by anti-oxLDL antibodies, but it should be understood that antibodies having other specificities can be produced in similar manner using other transgenes which produce particular in vivo modifications.

As indicated, the method for initially generating the antibodies uses a novel animal (e.g., mouse) strain. Monoclonal antibodies can be produced by hybridoma techniques or phage display (or other antibody display technique, e.g., yeast display) and screened for activity.

Focusing on anti-oxLDL antibodies, antibodies produced using hybridoma or a phage or cellular display technique can be screened against various forms of oxidized LDL (oxLDL) [Oxidized HDL (oxHDL) can also be used]. These in vivo-occurring antibodies to oxLDL (or oxHDL) provide detection of novel and sensitive biomarkers for acute coronary syndrome (ACS). Oxidized LDL and HDL play an important role in the early events leading to atherosclerosis and coronary artery disease. While several antibodies to oxLDL have been developed these have not proven useful to follow disease progression or predict an eminent cardiovascular event. One of the likely reasons is that a synthetic antigen was used to immunize mice for the production of monoclonal antibodies, which results in antibodies that do not detect all of the relevant forms of oxLDL and oxHDL. This leads to erroneous quantitation and misdiagnosis in patients.

Recently, the role of myeloperoxidase as an important component in the etiology and progression of atherosclerosis and coronary artery disease has been identified. In human atherosclerotic lesions, myeloperoxidase is expressed and oxidation products derived from myeloperoxidase are detected. In addition, elevated levels of myeloperoxidase in the serum of patients with an impending cardiovascular event are elevated. A novel mouse strain is advantageous to address this problem. Normally, in the low-density lipoprotein receptor deficient (LDLR−/−) mouse model of atherosclerosis, mice fed a high fat diet develop atherosclerosis but the mouse myeloperoxidase gene is not expressed in foam cell macrophages.

In a transgenic mouse in which the human myeloperoxidase gene has been inserted into the mouse genome and then crossed to the LDLR−/− mice, these mice not only develop atherosclerosis when fed a high fat diet but also express human myeloperoxidase in the atherosclerotic lesions and develop more oxidation products in these lesions. This is similar to what is observed in human atherosclerotic lesions. We use this novel mouse strain expressing human MPO to generate antibodies recognizing naturally occurring oxidized LDL and HDL which can be coupled to a sensitive assay system.

To summarize, the process of identifying the specific antibodies involves generating recombinant antibodies to oxidized human LDL and/or HDL from a novel strain of mice expressing human myeloperoxidase that has been crossed to the LDLR−/− mice. These mice develop severe atherosclerosis when fed a high fat diet and generate an antibody response to the in vivo oxLDL and oxHDL. Antibodies are screened against various forms of oxLDL and/or oxHDL to identify antibodies which specifically recognize one or more of the oxLDL or oxHDL forms.

In addition to the approach indicated above for generating and screening antibodies, in the case of oxLDL, antibodies can be generated using naturally occurring oxidized human LDL and/or HDL as immunogens. Recombinant antibodies can then be obtained through hybridoma and/or antibody display technologies. That is, human naturally occurring oxidized LDL and/or HDL is isolated from human plasma and used as an immunogen, e.g., in mice. The recombinant antibodies generated can be screened against various forms of oxidized human LDL and HDL, such as naturally occurring oxidized LDL and/or HDL as well as myeloperoxidase oxidized LDL and HDL.

### The Development of Atherosclerosis and the Role of Oxidation

Coronary heart disease (CHD) affects about 16.8 million people in the United States (estimates from 2006, American Heart Association). Oxidation of lipoproteins, particularly low-density lipoproteins (LDL) is believed to be an early event in the development of macrophage-derived foam cells. The oxLDL triggers the early development of atherosclerosis first through the recruitment of monocyte-derived macrophages into the arterial wall and second, by promoting the intracellular accumulation of cholesterol in these cells. Scavenger receptors on macrophages take up the oxLDL leading to foam cell formation [3]. In addition, oxLDL induces oxidative stress in endothelial cells, smooth muscle cells and macrophages resulting in a cycle of atherogenic plaque formation [4].

Cardiovascular heart disease takes years to develop and thus, developing biomarkers to track the disease is important for prognosis and prevention as well as evaluating preventative and therapeutic options. Given the role of oxLDL and oxHDL in the development of acute coronary syndrome the monitoring of oxLDL and oxHDL levels in human blood would be useful in the early diagnosis of acute coronary syndrome (ACS). The novel mouse model briefly described above is highly advantageous to develop the appropriate antibodies enables measurement of various forms of oxLDL and oxHDL.

### Myeloperoxidase is an Oxidant-Generating Enzyme Implicated in Atherosclerosis

Recent evidence implicates MPO in the generation of foam cell macrophages in early lesions, and in the progression to vulnerable plaques. MPO catalyzes a reaction between chloride and hydrogen peroxide to produce the potent oxidant, hypochlorous acid (HOCl), while also reacting with nitrite to generate reactive nitrogen species. MPO and its oxidant by-products, including 3-chlorotyrosine, are present in human atherosclerotic lesions, co-localizing with foam cell macrophages [5,6], and are especially abundant at sites of thrombosis [7]. Data from human and mouse studies have shown that MPO is involved in several aspects of lesion development and the risk of CAD events. MPO-oxidized LDL produces a more atherogenic form that is effectively taken up by scavenger receptors, CD36 and SR-A1, to give rise to foam cells. MPO preferentially oxidizes ApoA1, the major protein component of HDL, impairing ABCA-1 mediated cholesterol efflux from macrophages [8,9], further encouraging foam cell formation.

Increased levels of serum MPO correlates with increased risk of CAD events [10,11], while individuals with inherited MPO deficiencies have less cardiovascular disease [12]. An MPO promoter polymorphism, −463 G/A, which alters expression levels [13,14], has been associated with increased incidence of CAD [15-17] and severity of atherosclerosis [18,19] as well as higher serum lipids and body weight (BMI)[20,21].

Human MPO (huMPO) Transgenics Provide a Means for Testing the Role of MPO in Atherosclerosis

The mouse MPO is not expressed in the atherosclerotic lesions that develop in the LDLR−/− mice fed a high fat diet. To circumvent this problem, a human MPO transgenic mouse strain was developed [14]. The huMPO transgenics are a unique resource for investigating the regulated expression of huMPO in models of atherosclerosis. The mice were generated by injection into eggs of a 29 kilobase (Kb) DNA fragment containing the intact MPO gene with introns, along with several Kb of upstream and downstream flanking sequences.

The transgenics (2 lines) each have a single copy of the transgene, both of which have identical coding sequence. The transgenic mice produce functional MPO enzyme. When the huMPO transgenic mice were crossed to the LDL receptor deficient (LDLR−/−) mice and fed a high fat diet, the MPO transgene was expressed in aortic lesions [14]. The expression of the huMPO transgene correlated with increased atherosclerosis [1], protein oxidation in lesions [2], increased serum cholesterol and triglycerides, glucose, and weight gain/obesity [1], consistent with human studies [20,21].

### Antibodies to Oxidized LDL.

There have been a number of efforts over the last several years to identify antibodies to oxidized LDL [22]. It is well known that oxLDL is highly immunogenic and that there are increased autoantibody titers against epitopes of oxLDL correlating with increased atherosclerosis [23-25]. While it is known that oxLDL represents a variety of modifications of both lipid and protein components of LDL the majority of antibodies characterized so far have been against the lipid moiety, specifically oxidized phosphatidylcholine [26] [27] while another recognizes cross linkages between amino-lysine groups of apolipoprotein B and reactive aldehydes that result from the oxidation of long-chain fatty acids [28].

The availability of these antibodies has created considerable interest in the possibility that these could be used for diagnostic purposes. So far, while there have been hints that the antibodies may be useful, none of the currently available antibodies is used routinely to evaluate patients with acute coronary syndrome. There are a variety of possible reasons. Some prior studies identified antibodies to oxidized phosphatidylcholine, a major phospholipid in LDL, however these antibodies have not proven to be diagnostically relevant.

Second, the oxidation of LDL in vivo likely creates a number of epitopes, and the less abundant epitopes may be the more clinically relevant. The present approach, using a novel mouse model and hybridoma and/or antibody display technology (e.g., phage display) helps to resolve this issue by allowing us to identify and test a much larger number of antibodies including many recognizing less abundant epitopes, rather than limiting the analysis to the most abundant phospholipids.

Third, and most importantly, most of the monoclonal antibodies raised against oxLDL have been generated using non-physiological agents, e.g. copper sulfate, malondialdehyde (MDA), or 4-hydroxynonenal (HNE), which primarily affect the lipid moiety of lipoproteins. This raises the question as to whether the available antibodies recognize all stages of the atherosclerotic process and importantly, whether they recognize the diagnostically relevant forms of oxLDL.

Our approach is different from these earlier studies in that we are looking for antibodies against naturally occurring oxLDL and oxHDL in a mouse model in which there is oxidation contributed by human MPO, providing a novel approach to generate and characterize antibodies against oxLDL and oxHDL. Based on the evidence linking MPO to cardiovascular disease and our demonstration that oxLDL is a component of the atheroma in the progression of atherosclerosis through MPO generated oxLDL, generating antibodies against MPO oxidized epitopes on LDL and HDL will provide a series of antibodies that are useful as diagnostic tools for ACS.

In addition, using phage display to identify antibodies to naturally occurring oxLDL and oxHDL provides a major advantage in identifying antibodies that recognize the oxidation products. We estimate that our phage library contains about 10⁸ individual clones. Screening 10⁸ phage clones is very manageable and is several orders of magnitude greater than can be screened using traditional hybridoma technology.

Thus, the generation of these novel antibodies against in vivo-modified oxLDL and oxHDL provides a series of new reagents to evaluate serum from patients suspected of having cardiovascular disease along with existing risk factors and will greatly enhance the quality of oxLDL testing. The antibody identification can be further refined through continued characterization of the biologically relevant epitopes on oxLDL and oxHDL and further testing of the antibodies against more human serum samples to identify those antibodies that are best adapted to serve as diagnostic reagents for patients with ACS. It is possible and perhaps likely that, as with troponin I, several antibodies will be needed in an assay to better assess cardiovascular disease.

Approaches: As described above, two different approaches to generation of antibodies to oxLDL are described for the present invention. Particular applications of these approaches to oxLDL and oxHDL are described below.

Approach 1: Generate recombinant antibodies to oxidized human LDL and HDL from a novel strain of mice expressing human myeloperoxidase that has been crossed to the LDL receptor deficient mouse model. These mice develop severe atherosclerosis when fed a high fat atherogenic diet. There are two significant stages in this antibody identification approach. The first stage is to generate recombinant antibodies from MPO×LDLR−/− mice fed a high fat diet for five months. These mice develop severe atherosclerosis and generate antibodies specific to myeloperoxidase oxidized human LDL and/or HDL. The antibodies obtained from the mice are screened against various forms of oxidized LDL and/or HDL, thereby identifying antibodies having desirable specificities. A particular implementation of this approach is described below.

Stage 1: Generate Recombinant Antibodies from MPO×LDLR−/− Mice on a High Fat Diet for Five Months

In this first approach, a novel transgenic mouse that expresses human myeloperoxidase in a model of atherosclerosis is used. The advantage with this model is that naturally occurring antibodies to oxLDL and oxHDL are generated in response to the disease state.

The human MPO×LDLR−/− (huMPO-LDLR) mice develop antibodies to MPO oxLDL and oxHDL and the identification of these antibodies provides useful diagnostics. Advantageously single chain Fv (scFv) and/or Fab antibodies essentially as described [39] can be identified (as well as intact antibodies and/or other antibody fragments). The approach to the isolation of each is similar. Both versions can be applied and resulting antibodies compared as a diagnostic assay is constructed. The small size of the scFv proteins may make them more suitable as imaging agents while the Fab proteins may be more useful as a reagent for ELISA, for example.

The huMPOG-LDLR mice are typically fed a high fat diet (e.g., adjusted calories diet, 42% from fat, Harlan Teklad) for five months. By this time the mice will have developed atherosclerosis and express human MPO in the atherosclerotic lesions [1]. After sacrificing the animals the spleens are removed and RNA extracted. cDNA coding for antibody sequences is used to generate single chain variable fragment (scFv) or fragment, antigen binding (Fab) antibodies.

Two phage display systems were developed for single chain variable fragment (scFv) and fragment, antigen binding (Fab). For scFv the construct consists of: pelB-Vk-polyG linker-Vh-myc-polyH-glll. The pelB leader sequence is useful for directing the attached protein to the periplasmic membrane in E. coli. This is followed by the Vk immunoglobulin sequence plus a linker followed by the Vh immunoglobulin sequence. At the end of the construct are both His and myc tags. This is followed by the gene (glll) coding for filamentous phage protein III (plll) that, allows the antibody-plll fusion protein to be expressed on the surface of the phage. A THP terminator located 5′ to the CAP binding site prevents background transcription and leakiness of the Lac promoter. In the presence of glucose (1-2%) in the culture medium there is little transcription from this site. The kappa variable and heavy chain variable sequences can be amplified using specific primers and inserted into the phagemid.

For Fab the construct is similar to that for scFv consisting of: pelB-Vk-Ck-stop-AlP-Vh-Ch1-myc-PolyG-glll. The construct contains the Vk and Ck as well as the Vh and Ch1 regions. The other elements are basically the same.

The resulting scFv and/or Fab phage are used for panning. Biotinylated oxLDL or oxHDL are mixed with either scFv or Fab phage. These are bound to avidin-coated magnetic latex beads. Only the phage that binds to the oxLDL or oxHDL will be trapped while the phage that does not recognize the oxLDL or oxHDL will be washed away. After three rounds of panning, the high affinity binding phage are amplified. Colonies are picked and tested by ELISA for binding to oxLDL and oxHDL. Positive phage is used to infect phage competent cells (Maxim biotech) and free scFv and Fab are purified using IMAC columns.

Stage 2: The Antibodies are Screened Against Various Forms of Oxidized Human LDL and/or HDL.

Human LDL and/or HDL is isolated from human blood, e.g., by differential centrifugation as described [40,41]. Isolated fractions of the LDL (preferably 1.020 to 1.057 g/ml) and/or HDL (preferably 1.063 to 1.21 g/ml) prepared by differential centrifugation are dialyzed against phosphate buffered saline (PBS) containing 0.1 mM EDTA.

Human LDL has been separated into five subfractions and subfraction 5 was highly oxidized when isolated from hypercholesterolemic plasma but not normolipidemic plasma [42]. Subfraction five from hypercholesterolemic plasma is isolated as described [42]. Basically, after the LDL is isolated by density gradient centrifugation it is further fractionated by FPLC using a UnoQ12 column (BioRad). The level of oxidation can be estimated by assaying thiobarbituric acid-reactive substances (TBARS). Fraction 5 is used for panning to select for antibodies that recognize oxLDL. Fraction 5 from normolipidemic plasma can be used as a control.

Myeloperoxidase modified LDL and/or HDL can be generated essentially as described for screening of the phage clones [2,43]. While the main focus is to identify those antibodies that recognize epitopes generated as naturally occurring oxLDL and oxHDL, other methods exist to oxidize LDL and HDL. These methods can be used for comparison and as a filter for the antibodies that are generated. Copper sulfate is a common method used to oxidize LDL and HDL [41]. Malondialdehyde (MDA) is another oxidizing agent that is commonly used to oxidize LDL and HDL [41].

Advantageously the isolated antibodies can be tested against serum from patients with cardiovascular disease and compared against normal controls.

An advantageous criterion for selecting antibodies focuses on those antibodies that selectively recognize naturally oxLDL or oxHDL. Once the antibodies are selected they can be further evaluated using ELISA. Negative screens can include un-oxidized LDL and HDL. All of the antibodies can be classified based on the type of oxidation product they recognize. Thus, the antibodies can be screened against naturally occurring human oxidized LDL isolated as described above and various forms of MPO oxidized LDL and HDL. The antibodies can be further classified based on whether they recognize the same epitope by competition assays. The DNA of each clone can be sequenced to categorize the clones based on identical or very similar sequences.

Phage libraries have been generated displaying Fab′s from the spleens of huMPO-LDLR mice that were fed a high fat diet for 5 months. By panning various Fab′s recognizing different forms of oxLDL have been selected. For example, three clones isolated by panning against MDA oxidized LDL were tested against various forms of oxLDL by ELISA. Clones A10, G4 and G9 were all specific for MDA oxidized LDL. Clone A3 was a negative clone and BLK contained no phage as a control. The results show that the antibody response is specific to MDA oxidized LDL. These results demonstrate the ability to isolate naturally occurring antibodies from the spleens of the huMPO-LDLR mice using phage display. The antibodies are specific for particular forms of oxLDL and represent a small fraction of the antibodies that have been and can be isolated. The screening of this or other phage library against other forms of oxidized LDL as well as LDL from human serum can also be carried out.

Approach 2: Generate Monoclonal Antibodies Using Oxidized Human LDL and HDL as Immunogens.

In the second approach oxLDL and/or oxHDL from human serum is isolated and used to immunize animals, most commonly mice. The advantage here is that an immunogenic quantity of oxLDL or oxHDL can be delivered to the animal as a potent antigen.

Stage 1. Human Naturally Occurring oxLDL and oxHDL is Isolated from Hypercholesterolemic Human Plasma and Used as an Immunogen in Mice.

An alternative approach to obtaining naturally occurring antibodies to oxLDL and oxHDL will be to immunize mice with various forms of naturally occurring human oxLDL and oxHDL.

The human oxLDL and oxHDL can be isolated as described above. The oxLDL and oxHDL is then be used to immunize mice.

Stage 2. The Antibodies Generated are Screened Against Various Forms of Human Oxidized LDL and HDL.

scFv and/or Fab antibodies can be generated using the phagemid system described above. The antibodies are panned against both naturally occurring oxLDL and oxHDL as well as myeloperoxidase oxidized LDL and HDL. Non-oxidized LDL and HDL can be used as a control.

As the antibodies are identified that recognize naturally occurring oxLDL and/or oxHDL. Criteria as established in Approach 1 can be used for selecting the best antibodies.

### Further Developments.

The approach described for this invention is very effective to identify naturally occurring antibodies to oxLDL and oxHDL and these antibodies are useful as diagnostics to measure oxLDL and oxHDL in patients at risk for ACS. Once the antibodies are identified, they can be characterized and evaluated against serum from patients with different degrees of ACS. Selected antibodies can be used in a variety of assay formats, preferably ones which are capable of measuring analyte with increased sensitivity (in the nanogram to picogram range).

As an example, the assay system can utilize Time-Resolved Fluorescence (TRF). A benefit of TRF is that it combines the high sensitivity of fluorescence without incurring the expense and complexity of high specification filter sets. This implies replacing the conventional label by an alternative label that exhibits relatively long-lived fluorescence, permitting detection using temporal rather than spectral discrimination. An advantageous system can use a customized optoelectronic sensing system as well as inexpensive material such as nitrocellulose and inert microparticles that have very low or even undetectable nonspecific interaction with blood components permitting the measurement of oxLDL or oxHDL.

### LITERATURE CITED

1. Castellani, L. W., Chang, J. J., Wang, X., Lusis, A. J. and Reynolds, W. F. (2006) Transgenic mice express human MPO −463G/A alleles at atherosclerotic lesions, developing hyperlipidemia and obesity in −463G males. J Lipid Res.
2. Wang, Z., Nicholls, S. J., Rodriguez, E. R., Kummu, O., Horkko, S., Barnard, J., Reynolds, W. F., Topol, E. J., Didonato, J. A. and Hazen, S. L. (2007) Protein carbamylation links inflammation, smoking, uremia and atherogenesis. Nat Med, 13, 1176-84.
3. Glass, C. K. and Witztum, J. L. (2001) Atherosclerosis. the road ahead. Cell, 104, 503-16.
4. Galle, J., Hansen-Hagge, T., Wanner, C. and Seibold, S. (2006) Impact of oxidized low density lipoprotein on vascular cells. Atherosclerosis, 185, 219-26.
5. Daugherty, A., Dunn, J. L., Rateri, D. L. and Heinecke, J. W. (1994) Myeloperoxidase, a catalyst for lipoprotein oxidation, is expressed in human atherosclerotic lesions. J Clin Invest, 94, 437-44.
6. Hazen, S. L., Crowley, J. R., Mueller, D. M. and Heinecke, J. W. (1997) Mass spectrometric quantification of 3-chlorotyrosine in human tissues with attomole sensitivity: a sensitive and specific marker for myeloperoxidase-catalyzed chlorination at sites of inflammation. Free Radic Biol Med, 23, 909-16.
7. Sugiyama, S., Okada, Y., Sukhova, G. K., Virmani, R., Heinecke, J. W. and Libby, P. (2001) Macrophage myeloperoxidase regulation by granulocyte macrophage colony-stimulating factor in human atherosclerosis and implications in acute coronary syndromes. Am J Pathol, 158, 879-91.
8. Bergt, C., Pennathur, S., Fu, X., Byun, J., O'Brien, K., McDonald, T. O., Singh, P., Anantharamaiah, G. M., Chait, A., Brunzell, J., Geary, R. L., Oram, J. F. and Heinecke, J. W. (2004) The myeloperoxidase product hypochlorous acid oxidizes HDL in the human artery wall and impairs ABCA1-dependent cholesterol transport. Proc Natl Acad Sci USA, 101, 13032-7.
9. Zheng, L., Nukuna, B., Brennan, M. L., Sun, M., Goormastic, M., Settle, M., Schmitt, D., Fu, X., Thomson, L., Fox, P. L., Ischiropoulos, H., Smith, J. D., Kinter, M. and Hazen, S. L. (2004) Apolipoprotein A-I is a selective target for myeloperoxidase-catalyzed oxidation and functional impairment in subjects with cardiovascular disease. J Clin Invest, 114, 529-41.
10. Zhang, R., Brennan, M. L., Fu, X., Aviles, R. J., Pearce, G. L., Penn, M. S., Topol, E. J., Sprecher, D. L. and Hazen, S. L. (2001) Association between myeloperoxidase levels and risk of coronary artery disease. Jama, 286, 2136-42.
11. Brennan, M. L., Penn, M. S., Van Lente, F., Nambi, V., Shishehbor, M. H., Aviles, R. J., Goormastic, M., Pepoy, M. L., McErlean, E. S., Topol, E. J., Nissen, S. E. and Hazen, S. L. (2003) Prognostic value of myeloperoxidase in patients with chest pain. N Engl J Med, 349, 1595-604
12. Kutter, D., Devaquet, P., Vanderstocken, G., Paulus, J. M., Marchal, V. and Gothot, A. (2000) Consequences of total and subtotal myeloperoxidase deficiency: risk or benefit? Acta Haematol, 104, 10-5.
13. Piedrafita, F. J., Molander, R. B., Vansant, G., Orlova, E. A., Pfahl, M. and Reynolds, W. F. (1996) An Alu element in the myeloperoxidase promoter contains a composite SP1-thyroid hormone-retinoic acid response element. J Biol Chem, 271, 14412-20.
14. Kumar, A. P., Piedrafita, F. J. and Reynolds, W. F. (2004) Peroxisome proliferator-activated receptor gamma ligands regulate myeloperoxidase expression in macrophages by an estrogen-dependent mechanism involving the −463GA promoter polymorphism. J Biol Chem, 279, 8300-15.
15. Asselbergs, F. W., Reynolds, W. F., Cohen-Tervaert, J. W., Jessurun, G. A. and Tio, R. A. (2004) Myeloperoxidase polymorphism related to cardiovascular events in coronary artery disease. Am J Med, 116, 429-30.
16. Nikpoor, B., Turecki, G., Fournier, C., Theroux, P. and Rouleau, G. A. (2001) A functional myeloperoxidase polymorphic variant is associated with coronary artery disease in French-Canadians. Am Heart J, 142, 336-9.
17. Pecoits-Filho, R., Stenvinkel, P., Marchlewska, A., Heimburger, O., Barany, P., Hoff, C. M., Holmes, C. J., Suliman, M., Lindholm, B., Schalling, M. and Nordfors, L. (2003) A functional variant of the myeloperoxidase gene is associated with cardiovascular disease in end-stage renal disease patients. Kidney Int Suppl, S172-6.
18. Makela, R., Laaksonen, R., Janatuinen, T., Vesalainen, R., Nuutila, P., Jaakkola, O., Knuuti, J. and Lehtimaki, T. (2004) Myeloperoxidase gene variation and coronary flow reserve in young healthy men. J Biomed Sci, 11, 59-64.
19. Makela, R., Karhunen, P. J., Kunnas, T. A., Ilveskoski, E., Kajander, O. A., Mikkelsson, J., Perola, M., Penttila, A. and Lehtimaki, T. (2003) Myeloperoxidase gene variation as a determinant of atherosclerosis progression in the abdominal and thoracic aorta: an autopsy study. Lab Invest, 83, 919-25.
20. Hoy, A., Tregouet, D., Leininger-Muller, B., Poirier, O., Maurice, M., Sass, C., Siest, G., Tiret, L. and Visvikis, S. (2001) Serum myeloperoxidase concentration in a healthy population: biological variations, familial resemblance and new genetic polymorphisms. Eur J Hum Genet, 9, 780-6.
21. Makela, R., Dastidar, P., Jokela, H., Jaakkola, O., Saarela, M., Punnonen, R. and Lehtimaki, T. (2006) Relation of myeloperoxidase promoter polymorphism and long-term hormone replacement therapy to oxidized low-density lipoprotein autoantibodies in postmenopausal women. Scand J Clin Lab Invest, 66, 371-83.
22. Shaw, P. X., Horkko, S., Tsimikas, S., Chang, M. K., Palinski, W., Silverman, G. J., Chen, P. P. and Witztum, J. L. (2001) Human-derived anti-oxidized LDL autoantibody blocks uptake of oxidized LDL by macrophages and localizes to atherosclerotic lesions in vivo. Arterioscler Thromb Vasc Biol, 21, 1333-9.
23. Palinski, W., Tangirala, R. K., Miller, E., Young, S. G. and Witztum, J. L. (1995) Increased autoantibody titers against epitopes of oxidized LDL in LDL receptor-deficient mice with increased atherosclerosis. Arterioscler Thromb Vasc Biol, 15, 1569-76.
24. Tsimikas, S., Palinski, W. and Witztum, J. L. (2001) Circulating autoantibodies to oxidized LDL correlate with arterial accumulation and depletion of oxidized LDL in LDL receptor-deficient mice. Arterioscler Thromb Vasc Biol, 21, 95-100.
25. van Leeuwen, M., Damoiseaux, J., Duijvestijn, A., Heeringa, P., Gijbels, M., de Winther, M. and Tervaert, J. W. (2009) The IgM response to modified LDL in experimental atherosclerosis: hypochlorite-modified LDL IgM antibodies versus classical natural T15 IgM antibodies. Ann NY Acad Sci, 1173, 274-9.
26. Chang, M. K., Bergmark, C., Laurila, A., Horkko, S., Han, K. H., Friedman, P., Dennis, E. A. and Witztum, J. L. (1999) Monoclonal antibodies against oxidized low-density lipoprotein bind to apoptotic cells and inhibit their phagocytosis by elicited macrophages: evidence that oxidation-specific epitopes mediate macrophage recognition. Proc Natl Acad Sci USA, 96, 6353-8.
27. Itabe, H., Takeshima, E., Iwasaki, H., Kimura, J., Yoshida, Y., Imanaka, T. and Takano, T. (1994) A monoclonal antibody against oxidized lipoprotein recognizes foam cells in atherosclerotic lesions. Complex formation of oxidized phosphatidylcholines and polypeptides. J Biol Chem, 269, 15274-9.
28. Holvoet, P., Perez, G., Zhao, Z., Brouwers, E., Bernar, H. and Collen, D. (1995) Malondialdehyde-modified low density lipoproteins in patients with atherosclerotic disease. J Clin Invest, 95, 2611-9.
29. Katus, H. A., Remppis, A., Neumann, F. J., Scheffold, T., Diederich, K. W., Vinar, G., Noe, A., Matern, G. and Kuebler, W. (1991) Diagnostic efficiency of troponin T measurements in acute myocardial infarction. Circulation, 83, 902-12.
30. Bodor, G. S., Porter, S., Landt, Y. and Ladenson, J. H. (1992) Development of monoclonal antibodies for an assay of cardiac troponin-I and preliminary results in suspected cases of myocardial infarction. Clin Chem, 38, 2203-14.
31. Morrow, D. A., Cannon, C. P., Jesse, R. L., Newby, L. K., Ravkilde, J., Storrow, A. B., Wu, A. H., Christenson, R. H., Apple, F. S., Francis, G. and Tang, W. (2007) National Academy of Clinical Biochemistry Laboratory Medicine Practice Guidelines: clinical characteristics and utilization of biochemical markers in acute coronary syndromes. Clin Chem, 53, 552-74.
32. Wu, A. H., Feng, Y. J., Moore, R., Apple, F. S., McPherson, P. H., Buechler, K. F. and Bodor, G. (1998) Characterization of cardiac troponin subunit release into serum after acute myocardial infarction and comparison of assays for troponin T and I. American Association for Clinical Chemistry Subcommittee on cTnI Standardization. Clin Chem, 44, 1198-208.
33. Apple, F. S. (1999) Clinical and analytical standardization issues confronting cardiac troponin I. Clin Chem, 45, 18-20.
34. Tate, J. R. (2008) Troponin revisited 2008: assay performance. Clin Chem Lab Med, 46, 1489-500.
35. Panteghini, M., Bunk, D. M., Christenson, R. H., Katrukha, A., Porter, R. A., Schimmel, H., Wang, L. and Tate, J. R. (2008) Standardization of troponin I measurements: an update. Clin Chem Lab Med, 46, 1501-6.
36. Wu, A. H. and Jaffe, A. S. (2008) The clinical need for high-sensitivity cardiac troponin assays for acute coronary syndromes and the role for serial testing. Am Heart J, 155, 208-14.
37. Berridge, B. R., Pettit, S., Walker, D. B., Jaffe, A. S., Schultze, A. E., Herman, E., Reagan, W. J., Lipshultz, S. E., Apple, F. S. and York, M. J. (2009) A translational approach to detecting drug-induced cardiac injury with cardiac troponins: consensus and recommendations from the Cardiac Troponins Biomarker Working Group of the Health and Environmental Sciences Institute. Am Heart J, 158, 21-9.
38. Helfand, M., Buckley, D. I., Freeman, M., Fu, R., Rogers, K., Fleming, C. and Humphrey, L. L. (2009) Emerging risk factors for coronary heart disease: a summary of systematic reviews conducted for the U.S. Preventive Services Task Force. Ann Intern Med, 151, 496-507.
39. Hoogenboom, H. R. (2002) Overview of antibody phage-display technology and its applications. Methods Mol Biol, 178, 1-37.
40. Havel, R. J., Eder, H. A. and Bragdon, J. H. (1955) The distribution and chemical composition of ultracentrifugally separated lipoproteins in human serum. J Clin Invest, 34, 1345-53.
41. Palinski, W., Yla-Herttuala, S., Rosenfeld, M. E., Butler, S. W., Socher, S. A., Parthasarathy, S., Curtiss, L. K. and Witztum, J. L. (1990) Antisera and monoclonal antibodies specific for epitopes generated during oxidative modification of low density lipoprotein. Arteriosclerosis, 10, 325-35.
42. Chen, C. H., Jiang, T., Yang, J. H., Jiang, W., Lu, J., Marathe, G. K., Pownall, H. J., Ballantyne, C. M., McIntyre, T. M., Henry, P. D. and Yang, C. Y. (2003) Low-density lipoprotein in hypercholesterolemic human plasma induces vascular endothelial cell apoptosis by inhibiting fibroblast growth factor 2 transcription. Circulation, 107, 2102-8.
43. Zheng, L., Settle, M., Brubaker, G., Schmitt, D., Hazen, S. L., Smith, J. D. and Kinter, M. (2005) Localization of nitration and chlorination sites on apolipoprotein A-I catalyzed by myeloperoxidase in human atheroma and associated oxidative impairment in ABCA1-dependent cholesterol efflux from macrophages. J Biol Chem, 280, 38-47.

As used herein, the term “antibody” refers to in immunoglobulin or fragment thereof exhibiting specific binding properties. Unless clearly indicated to the contrary (e.g., by inclusion of the term “intact” or “fragment”), the term “antibody” includes intact antibodies (e.g., IgG immunoglobulin molecules) and antibody fragments and derivatives such as Fab, Fab′ (or F(ab′)₂), scFv, and sdFv, single chain antibodies such as those from camelids and sharks and fragments thereof such as VHH domain fragments, and hybrid antibodies containing regions derived from different organisms (e.g., humanized mouse antibodies).

A single chain variable antibody fragment (scFv) is a fusion protein containing variable heavy and variable light immunoglobulin sequences connected by a short linker.

An Fab (fragment antigen binding) antibody fragment is an antibody fragment consisting of a heavy chain variable domain and a light chain variable domain. Fab fragments may be obtained from intact antibodies by papain digestion.

An Fab′ or F(ab′)₂ antibody fragment is an antibody fragment containing two antigen-binding regions linked together below the immunoglobulin hinge region, typically through disulfide linkages.

A single domain antibody (sdAb) is an antibody fragment consisting of a single monomeric variable antibody domain. Like a whole antibody, it is able to bind selectively to a specific antigen. With a molecular weight of only 12-15 kDa, single domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). The first single domain antibodies were engineered from heavy chain antibodies found in camelids; these are called VHH fragments. Cartilaginous fishes also have heavy chain antibodies (IgNAR, ‘immunoglobulin new antigen receptor’), from which single domain antibodies called VICAR fragments can be obtained. An alternative approach is to split the dimeric variable domains from common immunoglobulin C (IgG) from humans or mice into monomers.

In connection with the present in vivo-modified biomolecules, the term “modified” refers to a chemical alternation of the biomolecule from its native state. Types of modifications include, without limitation, addition, removal, or alteration of one or more functional groups or atoms on the biomolecule (e.g., oxidation, reduction, acetylation, etc.), removal or addition of one or more subunits of a biomolecular chain (e.g., one or more amino acid residues of a polypeptide), and alterations to the backbone of a biomolecular chain.

An “in vivo modification” is thus a modification which occurs in vivo in an organism, for example, in vivo oxidation of LDL. In most cases, such in vivo modification results from in vivo biochemical reactions rather than from internal modifying agents such as penetrating radiation.

The term “modification epitope” is used herein to refer to an epitope in which the antibody binding includes one or more binding interactions with a modification site or a site which has undergone a three-dimensional change resulting from one or more modifications of the biomolecule.

As used herein, the term “antibody clone” refers to a purified cell line (e.g., hybridoma cell line), phage line (or other antibody-display line such as an antibody displaying yeast cell line) which produces an antibody (which may be an intact antibody or an antibody fragment). Of course, phage lines may be present in a suitable host cell, such as a suitable bacterial cell.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the selection of particular antibodies. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

What is claimed is:

1. A method for producing antibodies specific for in vivo-modified biomolecules, comprising
screening monoclonal antibody clones for specific binding activity to an in vivo modification epitope on a biomolecule or biomolecular complex, wherein said antibody clones are derived from a transgenic animal expressing a transgene, wherein expression of said transgene results in said in vivo modification or an increase in the level of said in vivo modification; and
identifying antibody clones having said specific binding activity.

2. The method of claim 1, wherein said biomolecule or biomolecular complex is low density lipoprotein (LDL).

3. The method of claim 1, where said transgene is human myeloperoxidase and said modified biomolecule is a component of oxidized low density lipoprotein (oxLDL).

4. The method of claim 1, wherein said transgenic animal is a transgenic mouse.

5. The method of claim 1, wherein said transgene is a human gene.

6. The method of claim 1, wherein antibodies binding specifically to said in vivo modification are selected from a plurality of hybridoma cell lines derived from said transgenic animal.

7. The method of claim 1, wherein antibodies binding specifically to said in vivo modification are selected by panning a plurality of clones in a phage display library.

8. The method of claim 1, wherein said selected antibody is an intact antibody.

9. The method of claim 1, wherein said antibody fragment is an Fab fragment, an Fab′ fragment, a scFv fragment, or a sdFv fragment.

10. The method of claim 2, wherein said antibody preferentially binds to a plurality of different forms of oxidized LDL, including at least one of mpo-LDL, MDA-LDL, and cu-LDL as compared to binding to unmodified LDL.

11. An isolated monoclonal antibody, comprising
a monoclonal antibody or antibody fragment derived from a transgenic animal expressing a transgene, wherein expression of said transgene results in in vivo modification of biomolecules and said antibody or antibody fragment binds specifically to an epitope containing at least a part of said in vivo modification.

12. The monoclonal antibody of claim 11, wherein said antibody comprises a variable sequence preferentially binding to a modification epitope resulting from expression of human myeloperoxidase in a non-human animal.

13. A kit for determining the presence or amount of oxidized LDL (oxLDL) in a sample, comprising
at least one antibody that specifically binds to an oxidized epitope in oxLDL, wherein said antibody is derived from a transgenic animal expressing human myeloperoxidase and preferentially binds to mpo-LDL rather than un-modified LDL.

14. The kit of claim 13, comprising a plurality of single-use aliquots of said antibody.

15. The kit of claim 13, comprising a plurality of lateral flow assay devices, wherein a single use aliquot of said antibody is present in each said assay devices.

16. A method for determining the presence or amount of oxidized LDL (oxLDL) in a sample, comprising
contacting said sample with at least one antibody which specifically binds to oxLDL, wherein said at least one antibody is derived from a transgenic animal expressing a transgene which results in in vivo oxidation of LDL; and
detecting binding of said at least one antibody to LDL as an indication of the presence or amount of oxLDL in said sample.

17. The method of claim 16, wherein said transgene is human myeloperoxidase.
